(19) United States
(12) Patent Application Publication
(10) Pub. No.: US 2018/0042475 A1
(43) Pub. Date: Feb. 15, 2018

(54) OPHTHALMOSCOPE

(71) Applicants: London School of Hygiene & Tropical Medicine, London (GB); University of Strathclyde, Glasgow (GB); Greater Glasgow and Clyde Health Board, Glasgow (GB)

(72) Inventors: Mario Ettore Giardini, Glasgow (GB); Nigel Bolster, Glasgow (GB); Iain Livingstone, Glasgow (GB)

(73) Assignees: London School of Hygiene & Tropical Medicine, London (GB); University of Strathclyde, Glasgow (GB); Greater Glasgow and Clyde Health Board, Glasgow (GB)

(21) Appl. No.: 15/560,787

(22) PCT Filed: Mar. 17, 2016
(86) PCT No.: PCT/GB2016/050737

§ 371 Date: Sep. 22, 2017

(30) Foreign Application Priority Data
Mar. 24, 2015 (GB) 1504915.8

(51) Int. Cl. A61B 3/12; A61B 3/14

(52) U.S. Cl. CPC A61B 3/12; A61B 3/14

(57) ABSTRACT

The invention relates to an adapter for modifying a mobile communication device such as, but not limited to, a smartphone, tablet computers or webcam to create an ophthalmoscope or fundus camera for ophthalmoscopy or fundus imaging comprising an illumination device, including component parts thereof; and a method of testing eye function and producing images using said camera and associated illumination device. The invention has application in both the medical and veterinary practices

### Field of the Invention

The invention relates to an adapter for modifying a mobile communication device such as, but not limited to, a smartphone, tablet computer or webcam to create an ophthalmoscope or fundus camera for ophthalmoscopy or fundus imaging comprising an illumination device, including component parts thereof; and a method of testing eye function and producing images using said camera and associated illumination device. The invention has application in both the medical and veterinary practices.

### BACKGROUND OF THE INVENTION

In 2010 the World Health Organization estimated that 39 million people worldwide are functionally blind, that 80% of these could either have been treated or prevented, and that 90% of preventable blind people live in low-income countries.

Therefore, the majority of blind people reside in low-income countries, where the least eye diagnostics and treatment resources are deployed. In such countries, there is a lack both of ophthalmic equipment and of trained personnel, thus preventing the appropriate early detection of potentially blinding health conditions and the consequent delivery of eye care.

Appropriate eye diagnostic equipment, designed for use by minimally trained personnel, within a cost range accessible to low-income countries, is therefore a strong and urgently felt need.

Also in higher income countries, eye disease is posing an increasing burden on health service, whether delivered by public or private providers. As the average population age increases, the financial, organisational and, ultimately, social burden of undertaking eye examinations impacts on the quality and scope of the eye healthcare services. Therefore, also in high- and middle-income countries, the availability of eye diagnostic equipment at a low cost and for operation by minimally trained personnel would be highly beneficial, by shifting eye disease screening and diagnostics from specialist to community healthcare settings.

In ophthalmic, neurological and general medical testing, ophthalmoscopy, which is the visualisation of the retina, is one of the key diagnostic techniques. It is performed routinely using tools known as “ophthalmoscopes” and “fundus cameras”. Thus as used herein, Ophthalmoscope is an instrument that allows a health professional to see the interior surface, or fundus, of the eye (ophthalmoscopy, funduscopy, fundoscopy, fundus viewing), and a fundus camera an instrument that takes a photograph or image of the interior surface of the eye.

Ophthalmoscopy is undertaken, in routine testing, through an instrument called a “direct ophthalmoscope”. It is a pen-sized (approximately 10-20 cm) viewing system held by a doctor in front of the patient's eye, often at very close face-to-face distance, typically within 5 cm. Such an instrument is simple, relatively inexpensive, yet difficult to use. In the western world, training is typically undertaken at undergraduate level within optometry and medicine. The field of view is very small (5 to 10 degrees at best), the aiming is critical and the focussing requires great manual dexterity. Moreover, the segmentation of the image in to very small fields requires the operator to look at a small portion of the retina at a time, and to reconstruct a “mental image” of the retina itself.

To overcome these limitations, a more expensive instrument can be used. It is called an “indirect ophthalmoscope”. It consists of a short-focal-length lens which the doctor holds in front of the patient's eye, and a headpiece, which carries a viewer that projects light through the lens, into the eye. The user aligns by hand the lens, between the eye and the viewer, and looks at the retina. The field of view is much wider than a direct ophthalmoscope (40+ degrees). However, the system is expensive and the use can be difficult due to the intrinsically delicate manual alignment. Proficiency in this technique is typically limited to post-graduate ophthalmology sub-specialist doctors.

Two further instruments are derived from the indirect ophthalmoscope: a fundus camera and a panoptic ophthalmoscope. In the “fundus camera”, an indirect ophthalmoscope is pre-aligned. The patient's head is immobilised through a head-and-chin rest, and a photograph is taken through the pre-aligned, indirect ophthalmoscope using a camera. The panoptic ophthalmoscope is a proprietary instrument. This instrument, effectively a monocular indirect ophthalmoscope, is pre-aligned, and held by a doctor as a single unit in front of the patient's eye. The user observes the retina through the instrument. A camera can be attached to the device.

These tools are expensive or difficult to use, or both.

Yet, the blinding pathologies, whose detection requires or benefits from ophthalmoscopy/fundus imaging, are many, most of them with high incidence and great societal relevance, and include, for example, glaucoma, age-related macular degeneration, diabetic retinopathy, some forms of stroke, retinal thrombo-embolic disease.

The availability of a simple, easy to use, inexpensive ophthalmoscope or fundus camera would therefore ideally provide a novel, potentially ground-breaking tool to meet the eye care needs outlined in the previous paragraphs. In this respect, mobile phones appear to offer a very attractive platform for ophthalmoscopy and fundus photography, as they are ubiquitous throughout both low- and high-income countries, are familiar to the wider population and, coupled with suitable adapters, allow visualisation, still image and video recording, and remote transmission of retinal images, often in association with further imaging and non-imaging based eye tests, all in turn implemented on the same phone platform. Platforms with the same widespread diffusion, also lending themselves to ophthalmoscopy, include tablet computers, also known as “tablets”, and webcams connected to a computer or tablet or phone. The techniques described in this patent can indeed be applied also to tablet computers and webcams.

Indeed, adapters have been described for this purpose.

Common issues with all ophthalmoscopy/fundus imaging adapters known in the literature are cost and complexity. Even the simplest and least expensive known adapter [M. E. Giardini, I. A. T. Livingstone, S. Jordan, N. M. Bolster, T. Peto, M. Burton, A. Bastawrous, A Smartphone Based Ophthalmoscope. Proceedings of the 36th Annual International Conference of the IEEE Engineering in Medicine and Biology Society, 2177-80 (2014)] reduces the cost and complexity (both of construction and of use) of an ophthalmoscope by sacrificing image quality and usability. Issues with such low-cost adapters are many. The adapter depends on the specific model of phone—i.e. an adapter designed for a phone of a given make and model cannot be moved to a phone of a different make and model. The adapter presents a corneal reflection—i.e. part of the light that illuminates the retina is seen in the retinal image captured by the phone as a flare or bright spot obstructing part of the retinal image. The adapter illuminates the field of view unevenly, and lateral shadows are present. The illuminator light backscatters into the camera lens, thus reducing the contrast.

All other adapters in the literature overcome these problems through increased cost and complexity with respect to said simplest and least expensive adapter.

In the present invention, we describe a mobile communication device such as a smartphone, tablet commuter or webcam adapter for ophthalmoscopy and fundus imaging that advantageously overcomes these limitations of the previous adapters on the market, and namely dependence on the mobile device model, presence of a corneal reflection, uneven field illumination, and light backscattering, yet maintaining a low cost and simplicity.

### Statements of Invention

According to a first aspect of the invention, there is provided an adapter for modifying a mobile communication device to create an ophthalmoscope or fundus camera for ophthalmoscopy or fundus imaging, respectively, comprising at least one illumination device for illuminating an eye of an individual prior to viewing same or taking at least one image of same; wherein said illumination device comprises at least one light channeling member adapted for directing light into the eye to be imaged characterized in that said light channeling member comprises at least one prism having contained therein or provided thereon at least one optical adjustment member.

Those skilled in the art will appreciate that the use of said prism allows light from said illumination source to be exquisitely directed into the eye to maximize the accuracy of use of the instrument. In certain embodiments a plurality of said prisms are used.

In a preferred embodiment of the invention said optical adjustment member comprises one or more regions adapted to perform at least one of the following functions: to act as a diffuser, for example to modify or increase the angle subtended by the illumination beam; to act as an absorber, for example to modify the angular or spatial distribution of the illumination intensity; to act as a polarizer either linear or circular polarizer, for example to modify the light polarization.

Advantageously, absorbing regions on or in the prism reduce unwanted reflections into the camera optics and/or shape the illumination beam so as to distribute illumination mostly over the fundus only and avoid excessive illumination of other parts of the image, such as other parts of the eye, and thus avoid over-exposure or automated exposure adjustments to the detriment of the fundus image's quality.

In a preferred embodiment of the invention said light channeling member comprises multiple prisms. This feature is particularly advantageous when anatomical landmarks are difficult to discern and/or where the illumination is uneven, such as in automated image analysis or in image stitching. Relevant examples of landmarks include the macula lutea and fovea centralis, which are relatively featureless anatomical areas, only discerned by subtle pigmentary/depth changes relative to the surrounding retinal landscape.

Furthermore, in prior inventions, a single light-channeling member, such as a prism, brings limitations when used in patients with moderate-to-high levels of ametropia (refractive error), where the ametropia causes a lateral shadow in the image. With multiple prisms the observed extenuated shadowing in ametropes is circumvented, representing a significant expansion of utility in high myopes (near-sightedness) and high hypermetropes (long-sightedness).

In a further preferred embodiment of the invention said illumination device comprises a light source. More ideally, said light source is positioned off-center with respect to the eye pupil and/or the axis of the retinal imaging optics, with a light entry point off-centre by at least 0.1 mm, ideally 0.5 mm being more typical (this provision of off-centre light source is limited only by the pupil diameter) but up to and including 4 mm. In a further preferred embodiment of the invention we compensate for this off-centering by employing a divergent, ideally non-focused, illumination beam which also, ideally, is on an axis tilted by a few degrees (range: 1-20 degrees, including all 0.01 degree intervals there between, 5 degrees being more typical) away from the optical imaging axis. The afore light source arrangement, advantageously, reduces corneal reflection whilst simultaneously reducing shadowing and improving the field homogeneity.

In yet a further preferred embodiment of the invention and said light channeling member comprises a miniature optical fibre attachment. Preferably, when referring to a prism or a miniature optical fibre attachment the size under consideration is in the order of 1×1×2 mm up to 1×1×30 mm. This, advantageously, ensures the working distance can be as low as 1-2 mm.

In yet a further preferred embodiment of the invention said prism(s) is/are further provided with at least one reflective member positioned so that light exiting from said prism(s) is reflected towards said eye. Ideally, said reflective member is located towards the rear of the prism(s) or away from said eye. In yet a further preferred embodiment of the invention said reflective member is located on a first side of said prism(s) and, more ideally still, on a first and a second side of said prism(s) whereby light exiting from said prism(s) is reflected towards said eye.

In a further preferred embodiment of the invention said optical fibre attachment is a waveguide with at least one opening positioned so that, in use, light exiting from said waveguide is directed via said prism(s) towards said eye. More preferably, said waveguide comprises a plurality of openings and, ideally, has a scattering structure, ideally but not exclusively, made from corrugations, frosting, or the inclusion of particles. Other scattering surfaces or re-emissive surfaces (e.g. by fluorescence) will be well known to those skilled in the art and may be used in the working of the invention.

In a further preferred embodiment of the invention said waveguide is provided with at least one reflective member whereby light exiting from said waveguide is reflected via said prism(s) towards said eye. Ideally, said reflective member is located towards the rear of the waveguide or away from said eye. In yet a further preferred embodiment of the invention said reflective member is located on a first side of said waveguide, and more ideally still, on a first and a second side of said waveguide whereby light exiting from said waveguide is directed via said prism(s) towards said eye.

In yet a further preferred embodiment of the invention said illumination device comprises, either as part of said prism(s) and/or independently thereof, at least one circular polarizer positioned in the path of light that has to enter the eye (an illumination polarizer) whereby, before entering the eye, the light is circularly polarized. This arrangement reduces corneal reflection. Preferably, a film polariser is used in order to maintain low costs. In yet a further preferred embodiment a second circular polariser, ideally with the same or substantially the same polarisation chirality as the illumination polarizer, and ideally but necessarily mounted in front of the camera lens, is provided to block any specular reflection, including the corneal reflection. Again, a film polariser may be used for this purpose. Alternatively, two sections of the same circular polarizer may be employed as the illumination and second camera polarisers

In yet a further preferred embodiment of the invention said light source is either the device's own flash, or an independently powered light source such as, for example, a white or coloured LED. Where the light source is independent of the device it is adapted to be powered using the phone's Universal Serial Bus (USB) or earpiece jack, both present on most phones, alternatively, other proprietary connectors such as USB-on-the-go or a solar panel, are used thus rendering the operation of the light source independent of the phone this enables a user to control the intensity of the light, when it is turned on and off, e.g. either asynchronously or synchronously with the image acquisition, or turning on/off/synchronising multiple light sources.

In yet a further preferred embodiment of the invention said light source is a Light-emitting diode (LED), Organic LED (OLED), a flame, a fluorescence emission, an electric discharge in a gas, a conventional incandescent lamp, a halogen lamp, a laser, or sunlight/daylight.

In yet a further preferred embodiment of the invention light of one colour may be used e.g., blue or ultraviolet and the channeling member is made, at least in part, of a material emitting the desired light spectrum (e.g. white, or red-free) in this instance a suitable material would be a fluorescent material.

In one embodiment of the invention, particularly where the light source is an LED or lamp, said light channeling member takes the form of at least one reflective surface positioned at least partially about or adjacent said light source whereby light is directed into the eye to be photographed.

In yet a further preferred embodiment of the invention a plurality of light sources are provided and arranged so as to illuminate the eye from a plurality of different directions. Advantageously, this arrangement ensures a reduction in the unevenness of the retinal illumination (for example, less or more illuminated spots, shadows). Accordingly, in this preferred embodiment a plurality of light channeling members are provided or a single multi-faceted light channeling member is used that conveys light from different directions simultaneously. In either embodiment said light channeling member(s) comprise(s) said prism having contained therein or provided thereon at least one optical filtering or optical absorbing region. In one example of the invention, the single multi-faceted light channeling member is provided by placing an illuminator ring, line, circular or linear or other curved segment, around the camera.

In all these configurations at least one illumination polariser is provided per light source or a single illumination polarizer is shared amongst multiple illumination devices.

In all these configurations, the light channeling members draw light from the same light source or from multiple light sources.

In yet a preferred embodiment of the invention said channeling member also, advantageously, blocks scattered light from entering said camera.

In yet a further preferred embodiment of the invention said camera is a webcam or a mobile phone camera, digital camera, film camera, or camera of a tablet, netbook, thin client or laptop computer. Ideally it is a smartphone camera, either autofocussing or with long depth of field as present on the phone. Once the retina is illuminated by said light source the camera looks into the eye pupil, thus imaging the retina. Preferably focussing of the retina on the image is achieved by the native phone focussing, or long depth of field.

Advantageously, where the light source is an LED, or some other electrically powered source it can, optionally, be powered by the sound output jack of the phone/computer running the camera. Typically the electrical waveform generated at the sound output is fed to the light source, after optional rectification.

In a further preferred embodiment of the invention the illumination device or light source is separated from the imaging system in the direction of the imaging system's optical axis; this reduces shadowing by increasing the illuminator's field-of-view relative to the imaging system's field-of-view. This is of particular advantage where it is possible to place the illuminator closer to the eye than it is to place the imaging system, for example a smartphone camera whose working distance is limited by the size and geometry of the phone in which it is integrated, thus reducing shadowing by increasing the field-of-view of the illuminator without decreasing the field-of-view of the imaging system.

In yet a further embodiment of the invention the adapter is arranged so that the intensity of light stimulating the eye or the field diameter of the light stimulating the eye can be adjusted in order to allow a consistent pupil size to be maintained during non-mydriatic use where the pupil diameter is not fixed pharmacologically. Such an adjustment is advantageous to image particular structures of the eye or for a particular field-of-view (e.g. the optic nerve typical requires approximately 5 degree field-of-view). This field of view varies with pupil diameter which, at a given photopic luminance and field diameter, vary from person to person, primarily as a function of age.

In a preferred embodiment of the invention the adapter is provided with a control device allowing a user to vary the stimulating photoptic luminance between 10 and 10,000 candela per metre squared, including all one unit intervals there between, but most typically between 200 and 4000 candela per metre squared, including all one unit intervals there between or with the ability to adjust the luminance delivered across a scale of discrete units. This is particularly advantageous for use in non-mydriatic context where the pupil diameter is not fixed pharmacologically.

Those skilled in the art will appreciate that the said control device may reduce the delivered luminance by extinguishing or dimming one or more light sources, moving one or more light sources mechanically, introducing or moving optical components so as to change the direction, intensity or dispersion of the light directed towards the eye, or to direct one or more sources away from the eye altogether.

Conversely, those skilled in the art will appreciate that the said control device may increase the delivered luminance by lighting or increasing the brightness of one or more light sources, moving one or more light sources mechanically or by introducing optical components so as to change the direction, intensity or dispersion of the light directed towards the eye, or to guide one or more sources towards the eye where said source had previously been directed away from the eye.

In a further preferred embodiment of the invention said adapter includes a light source, or sources, that are pulsed or, otherwise vary in intensity with time, in order to delay the pupillary reflex.

Preferably, the adapter comprises a clip adapted to be attached to a phone, thus making it independent from the specific shape of the phone.

According to a further aspect of the invention, there is provided an ophthalmoscope or fundus camera comprising a mobile communication device having an integral camera and at least one associated illumination device for illuminating an eye of a patient prior to viewing same or taking at least one image of same; wherein said illumination device comprises at least one light channeling member adapted for directing light into the eye to be imaged characterized in that said light channeling member comprises at least one prism having contained therein or provided thereon at least one optical adjustment member.

Those skilled in the art will appreciate that where the mobile communication device has or includes a camera our invention enables the creation of an ophthalmoscope and/or a fundus camera and where the mobile communication device does not include a camera our invention enables the creation of an ophthalmoscope.

According to a third aspect of the invention there is provided a method for visualising the retina through the pupil (ophthalmoscopy) of an individual involving the use of the ophthalmoscope of the invention.

According to a fourth aspect of the invention there is provided a method for imaging the interior of the eye (fundography, fundus photography) of an individual involving the use of the fundus camera of the invention.

In use, we take an automatic-focus small camera, such as a good webcam or a good mobile phone camera (we typically use the whole phone, without modifications). We use the autofocussing feature of the camera to compensate for viewing defects (ametropies). We inject light into the eye by using in front of the camera either an appropriate miniature prism or a miniature optical fibre attachment. We then move the camera very close to the eye, effectively using the pupil as a window onto the retina. This is the principle currently used in direct ophthalmoscopy. However, the very small size of our illuminating device as well as the small size of the front lens of the autofocussing camera, allows us to move very close to the eye itself, thus expanding the field of view. In fact, we obtain a field of view approaching the field of an indirect ophthalmoscope, with a resolution comparable to the best fundus cameras, and superior to contemporary techniques for preterm/infant children.

Advantageously, the image quality is superb and the ease of use is such that minimally trained non-specialist operator can use the instrument after only a few minutes of instructions. The instrument has the potential to replace standard, bulky indirect ophthalmoscopy, panoptic ophthalmoscopy and retinal imaging through fundus cameras within a very small amount of time. Moreover, the instrument allows untrained personnel to take images in-field (such as in low-income countries, in prisons, in aerospace settings, in scientific expeditions, etc.) and to relay them easily and directly to an analysis point (for example, hospital, ophthalmic practice, retinal image analysis centre or automated retinal image analysis server), e.g. for screening, or for emergency or remote diagnostics. Moreover, with the use of appropriate software the instrument allows relatively untrained personnel to make a diagnosis using a captured image. Future use cases may include self-examination, with links to automated screening algorithms.

According to a further aspect of the invention there is provided a prism or a plurality of prisms, in or for use in a mobile communication device adapter, wherein said prism(s) has contained therein or provided thereon at least one optical adjustment member.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

An embodiment of the present invention will now be described by way of example only with particular reference to the following wherein:

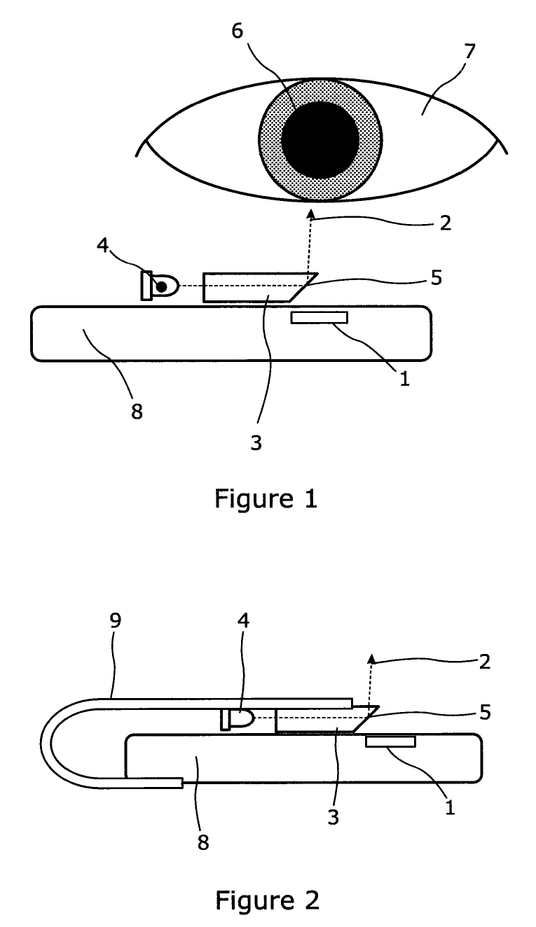

FIG. 1 shows a diagrammatic representation of an imaging system showing the various components;

FIG. 2 shows a diagrammatic representation of how the imaging system of FIG. 1 is attached to a smartphone;

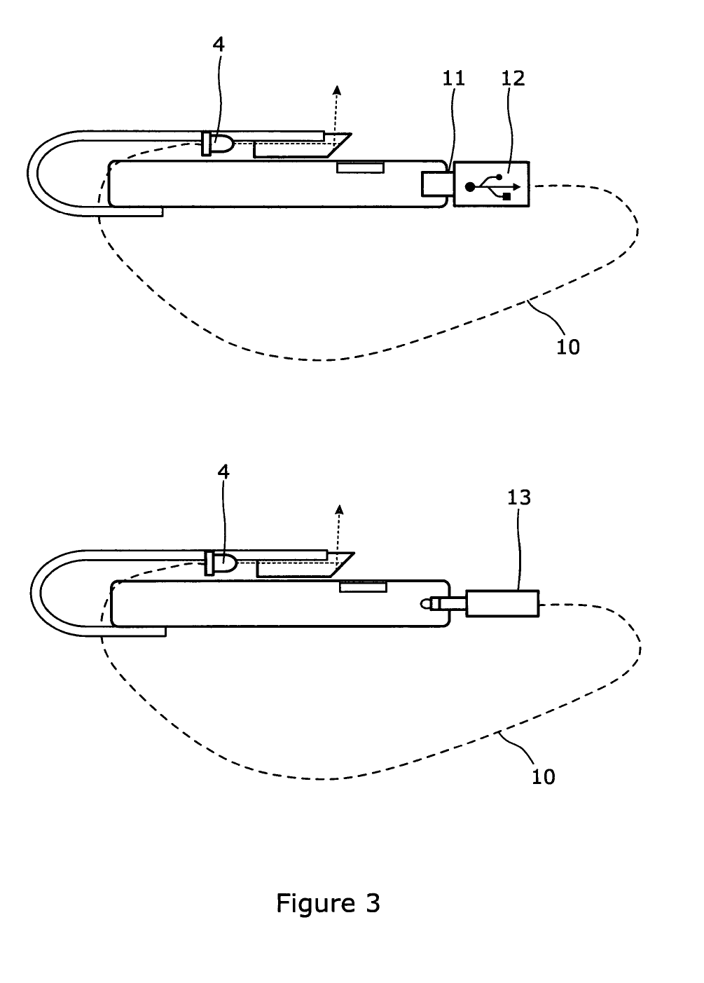

FIG. 3 shows a diagrammatic representation of how the system is powered;

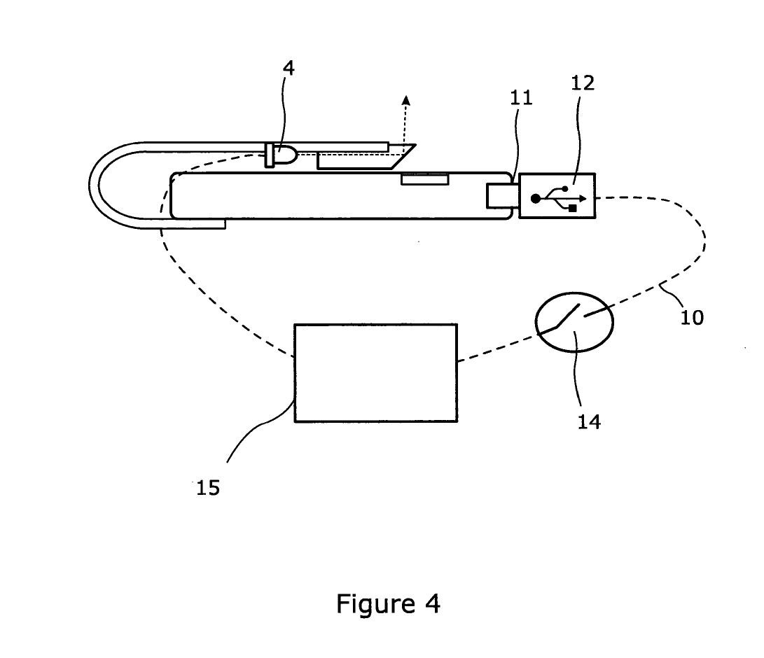

FIG. 4 shows a diagrammatic representation of how the powering of the system is controlled;

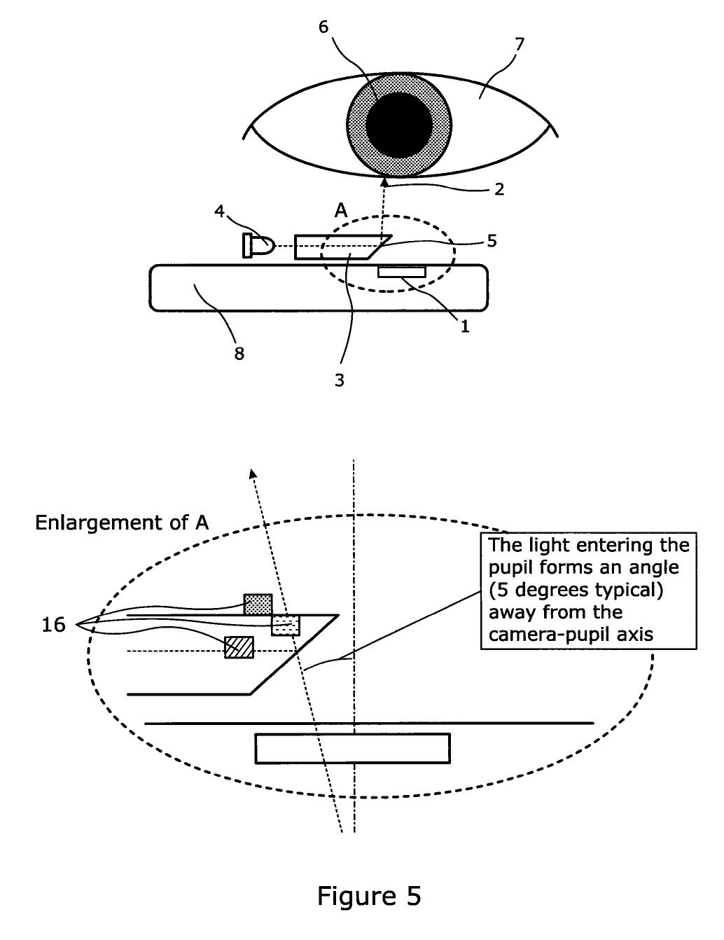

FIG. 5 shows a diagrammatic representation of how focussing of the light is achieved;

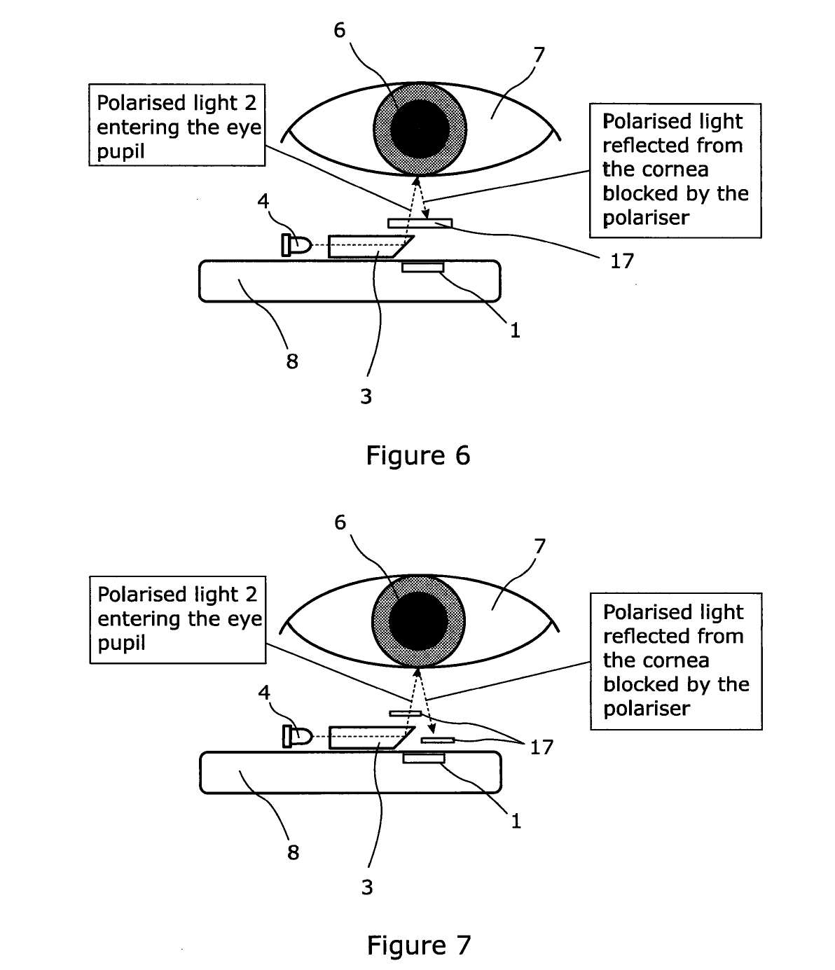

FIG. 6 shows a diagrammatic representation of how corneal reflection is blocked;

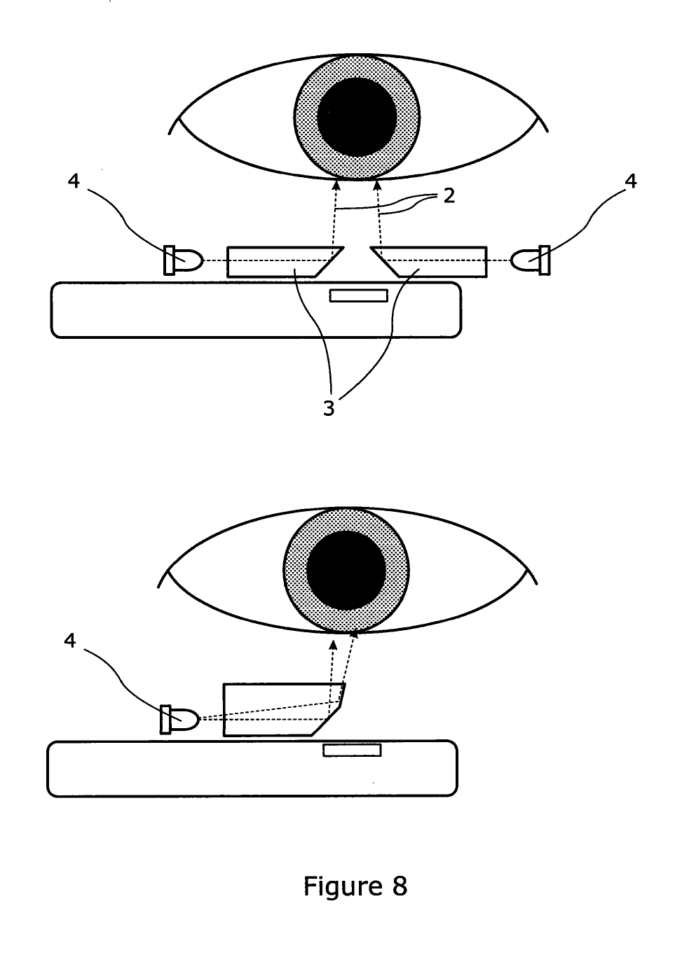

FIG. 7 shows a diagrammatic representation of how in a further alternative embodiment corneal reflection is blocked;

FIG. 8 shows a diagrammatic representation of the use of multiple illumination sources;

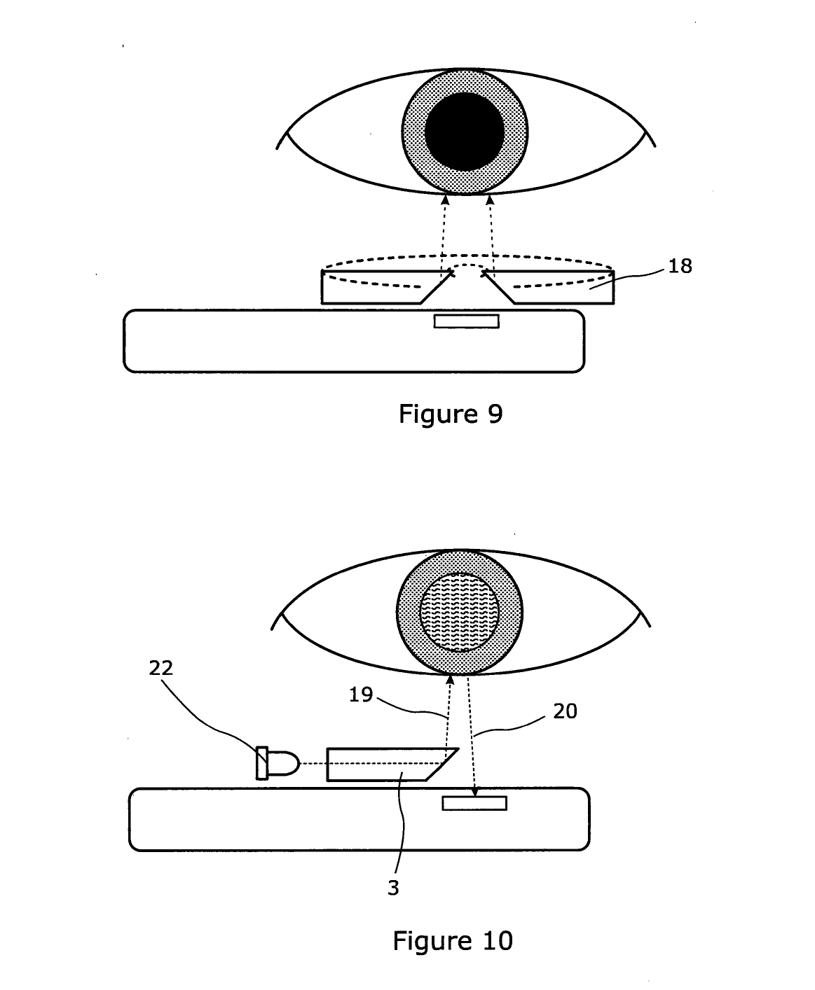

FIG. 9 shows a diagrammatic representation of the use of a single illumination source;

FIGS. 10 shows a diagrammatic representation of a further embodiment of the invention using a single or dual wavelength light source;

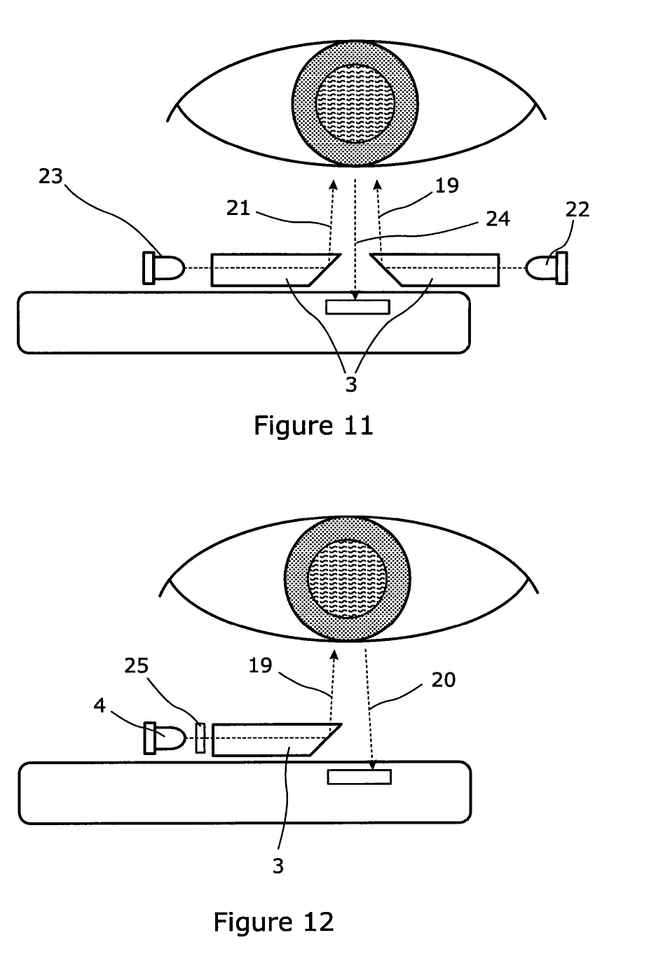

FIG. 11 shows a diagrammatic representation of a further embodiment of the invention using a single or multiple wavelength light source;

FIG. 12 shows a diagrammatic representation of a further embodiment of the invention using a fixed colour filter;

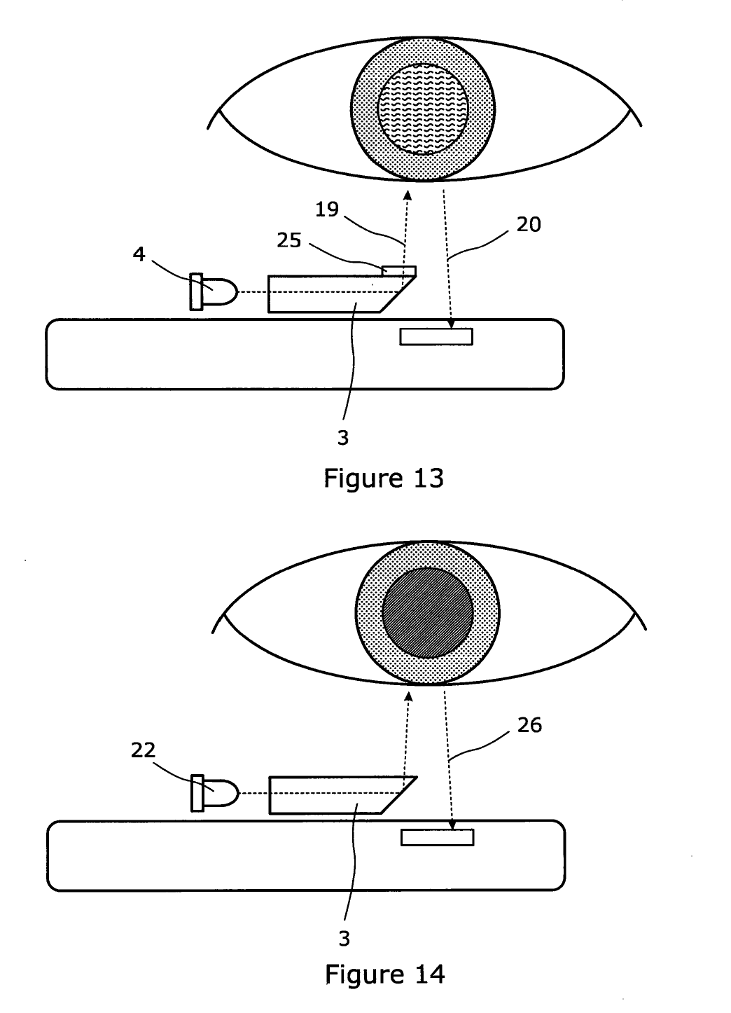

FIG. 13 shows a diagrammatic representation of a further embodiment of the invention using a fixed colour filter;

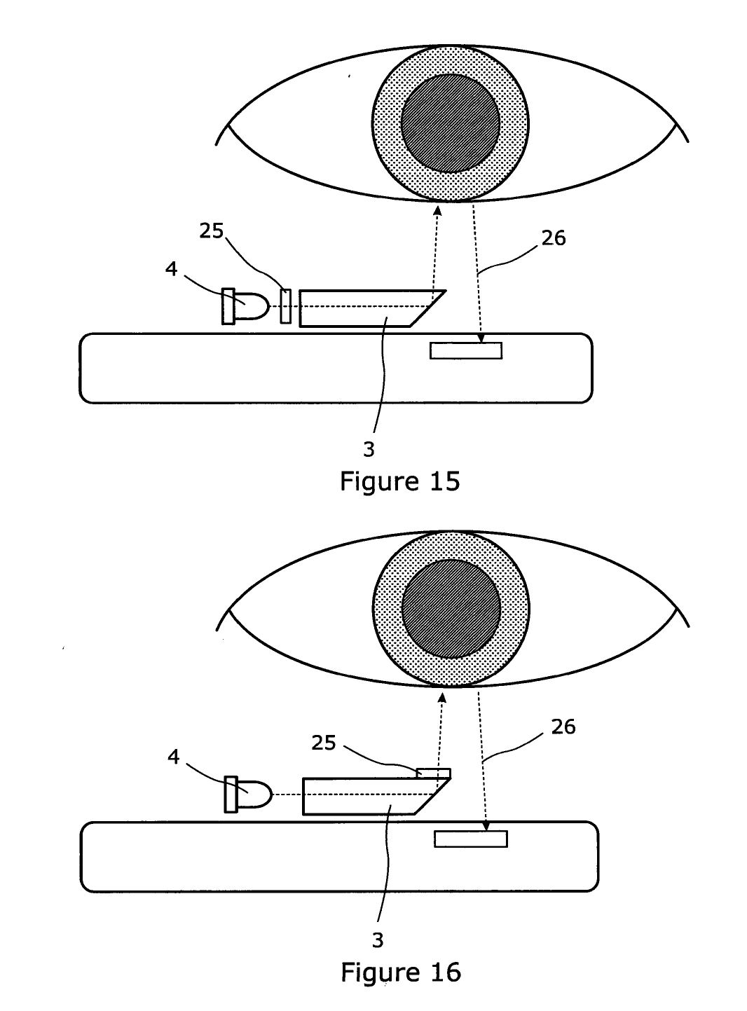

FIGS. 14-17 show diagrammatic representations of further embodiments of the invention wherein selected wavelength(s) of reflected light are used to obtain information about the eye;

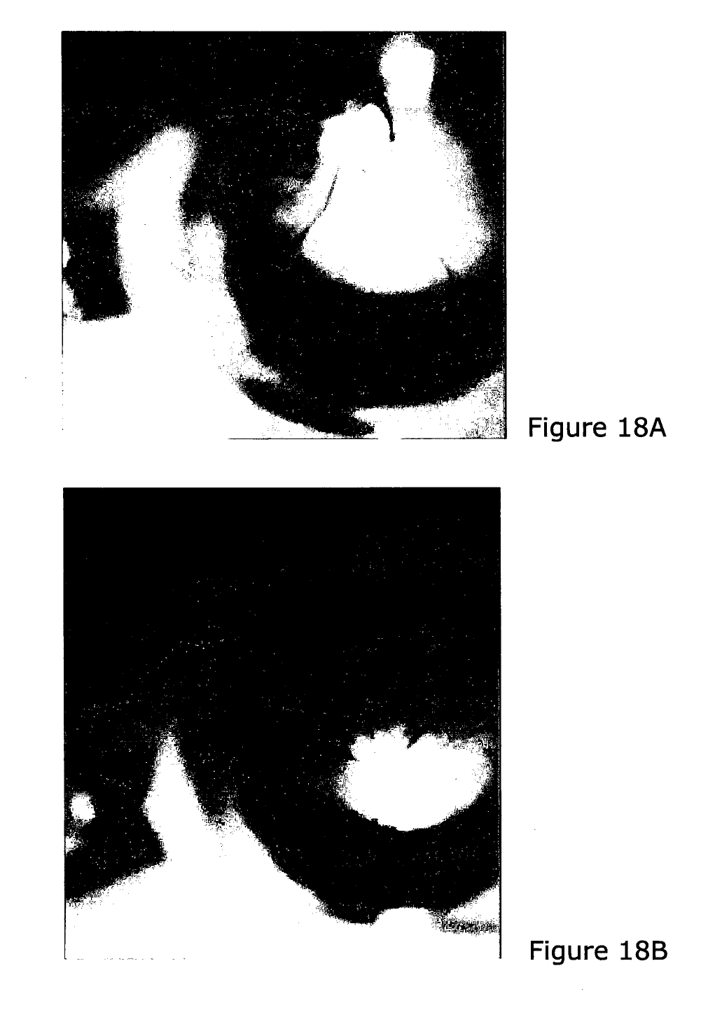

FIG. 18a is a retinal image showing corneal reflection when no polarizers are used, 18b shows the corneal reflection is strong and big enough to obscure the entire optic disk;

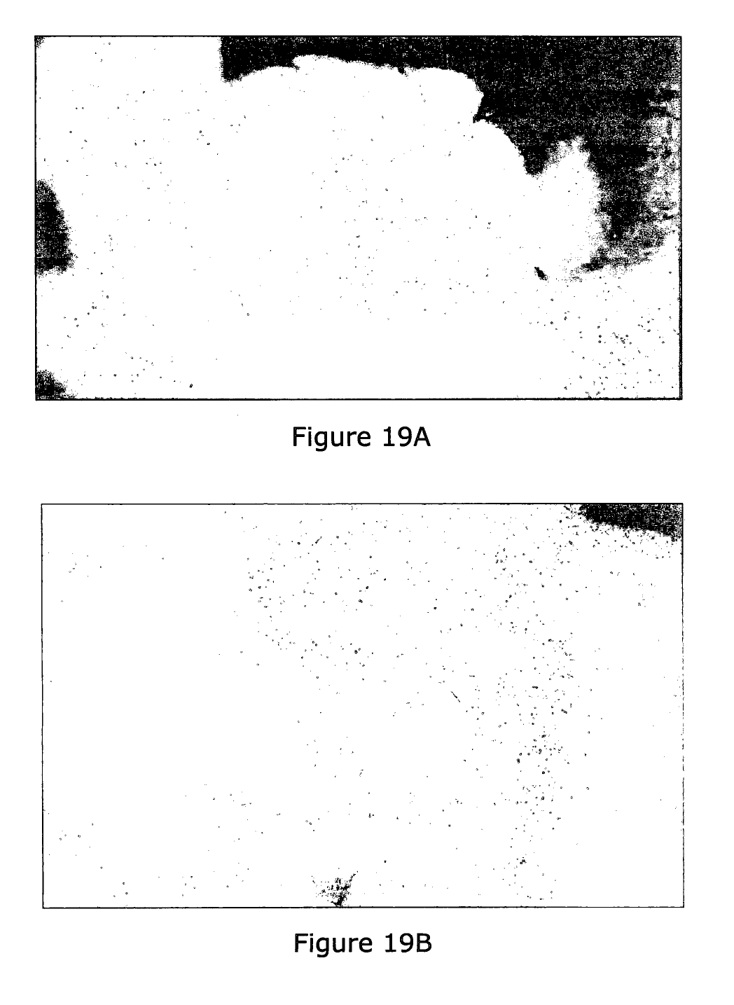

FIG. 19 shows the fundus image of the same eye shown in FIGS. 10a and 10b that is achieved with the addition of split polarizing filters, the corneal reflection is barely visible and does not significantly obscure fundus structures;

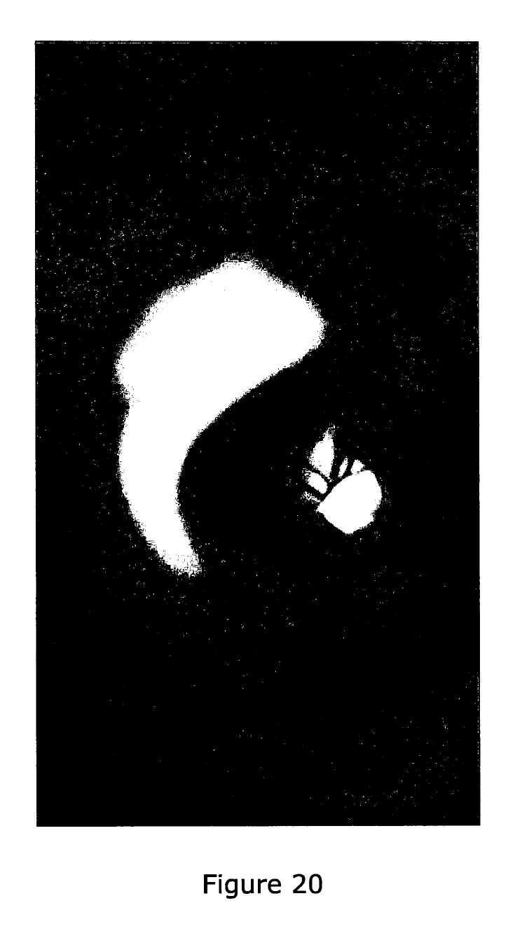

FIG. 20 is a retinal image showing how tilting the illumination source with respect to the optical axis allows the area of retinal illumination to be tuned for best illumination at angles of specific interest, such as the optic nerve;

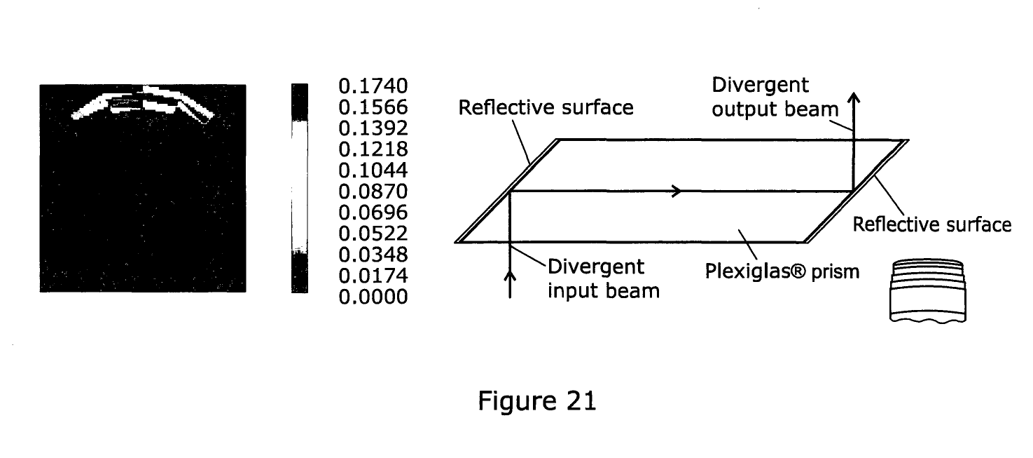

FIG. 21 shows how shadow is reduced through tilting the optical axis of the light source, this is simulated by placing a hem i-spherical reflector behind an aperture and arranging an illumination source and detector with a small lateral separation; a 0 degree tilt of the source optic axis relative to that of the detector casts nearly the entire sensor in shadow false colour image, left, intensity is according to various shades of grey;

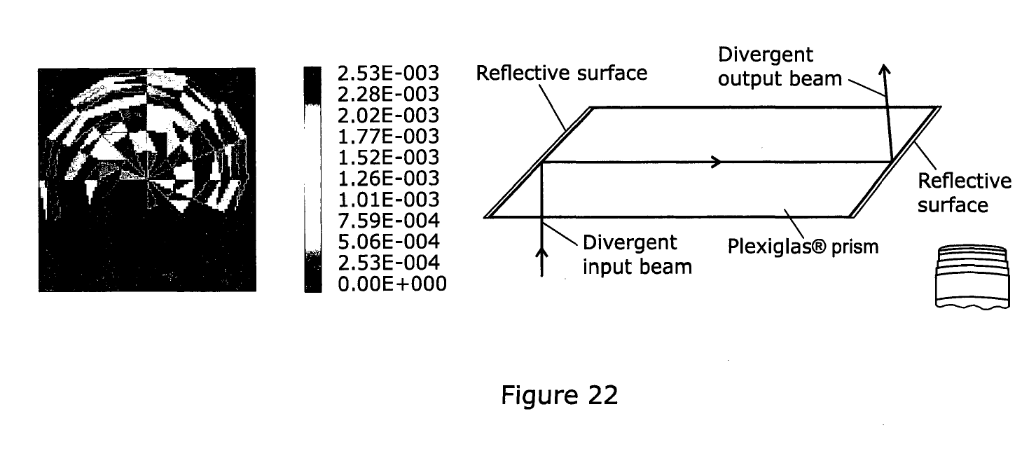

FIG. 22 shows the same arrangement with a 5 degree tilt of the source optic axis relative to that of the detector. The light falling on the detector (false colour image, left, shows intensity according to various shades of grey) is much more evenly distributed and the shadow is markedly reduced;

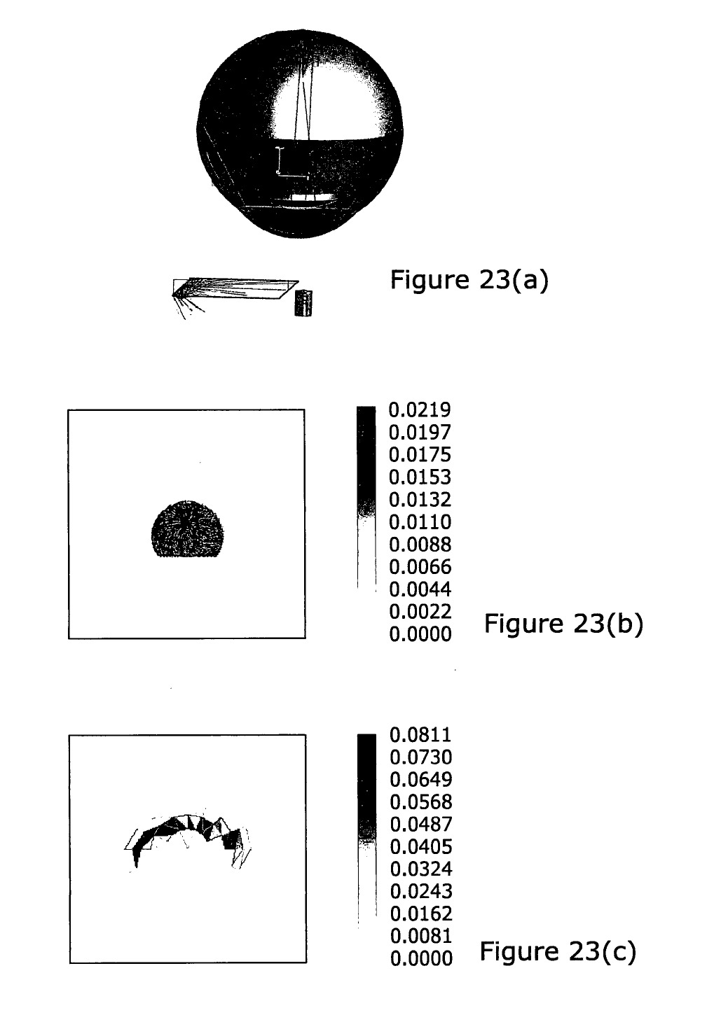

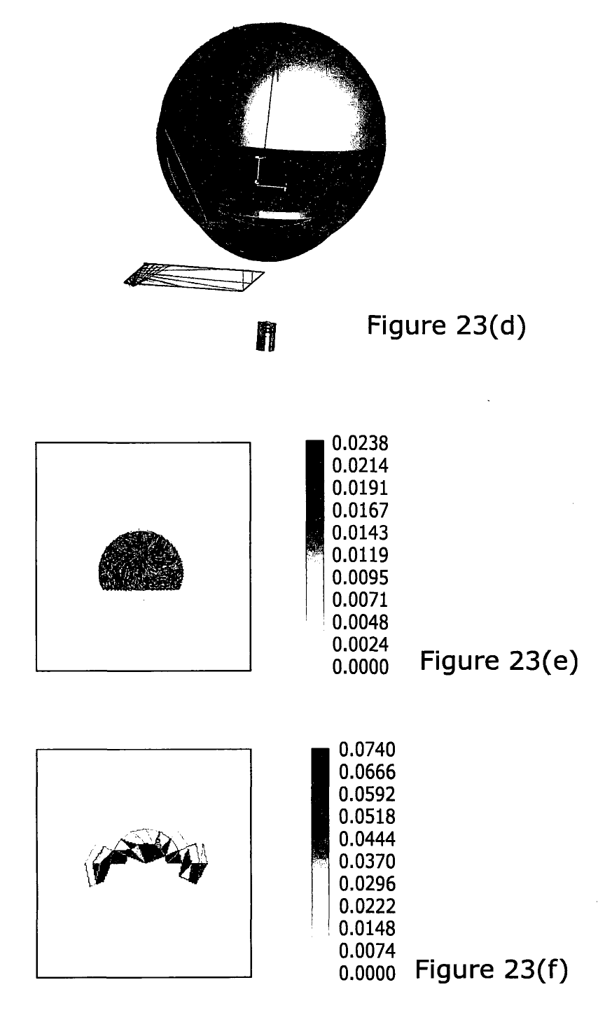

FIG. 23 shows

(a) an embodiment of the invention wherein the configuration has the channelling member's centre being coplanar with detector optics' front face

(b) False-greyscale image of the illumination distribution on the fundus of a normal adult eye when the configuration in 23(a) is used.

(c) False greyscale of the fundus image formed by the detector optics when the configuration in 23(a) is used.

(d) an embodiment of the invention wherein the configuration has the channelling member centre being 4mm closer to the eye than the front face of the detector optics.

(e) False-greyscale image of the illumination distribution on the fundus of a normal adult eye when the configuration in 23(d) is used. Area is to the same scale as 23(b)

(f) False greyscale of the fundus image formed by the detector optics when the configuration in 23(d) is used. Area is to the same scale as 23(c);

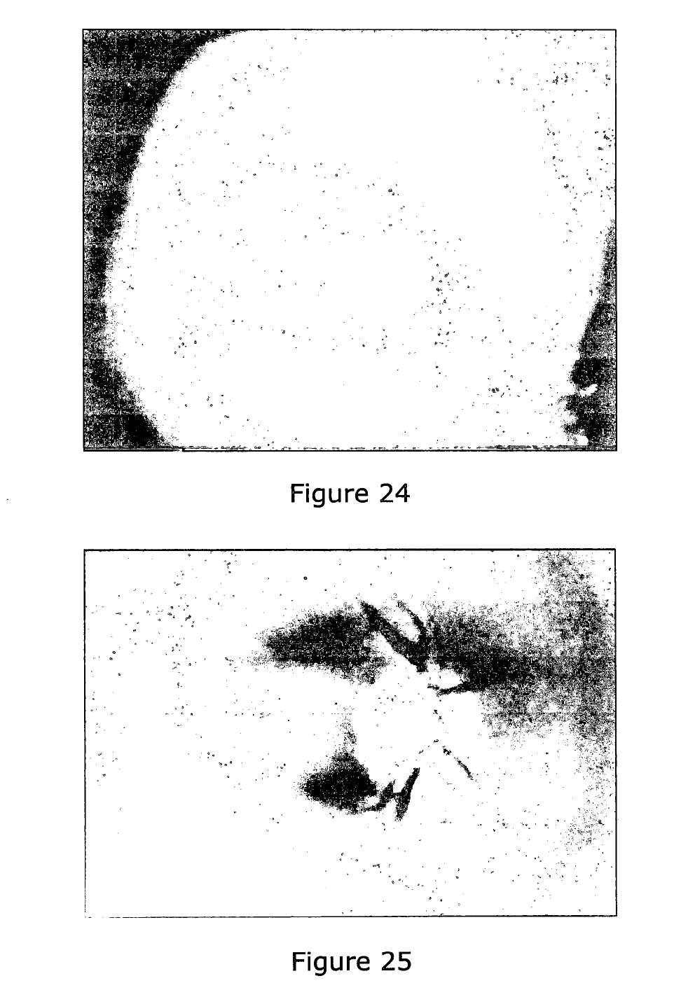

FIG. 24 shows an image acquired using two sources of the same intensity on a non-mydriatic eye. The field-of-view acquired is only just that at which the optic nerve can be viewed; and

FIG. 25 shows an image acquired using the same device as used to take the image shown in FIG. 24, but with one of the two sources disabled. The pupil of the eye is larger than in FIG. 24 and therefore more of the fundus is visible around the optic nerve.

Referring firstly to FIG. 1 there is shown a schematic representation of an ophthalmoscope or fundus camera in accordance with the invention. An imaging system (such as a webcam, mobile phone camera, digital camera, film camera, web cam or tablet camera,) is shown as 1, viewing in direction 2 into the pupil 6 of the eye under observation 7. A light channeling member 3 (in this embodiment a single prism) guides light from a source 4 to a prism head indicated at 5. The light is ‘guided out’ of the prism by total internal reflection from or metallisation of the prism, into the pupil 6. The prism size is such that the camera can be very close to the pupil 6 of the eye under observation 7, thus maximising the field of view. The size of the prism is typically, but not exclusively, within the range 1×1×1 mm up to 10×10×30 mm, including all 1mm variations in between of height, width and depth.

The light source 4 is in the form of a lamp, inorganic light-emitting diode (LED), organic light-emitting diode (OLED), flame, sun, moon, stars, incandescent metal, chemical reaction, heated surface, laser, fluorescent or phosphorescent material.

In all of the figures, the light source is divergent and unfocussed. Indeed our device, unlike the state of the art, does not require any focussing or convergence of the light source in order to perform any of the functions or to implement any of the benefits or technical solutions described.

In a single embodiment of the invention the imaging system and the light source is, respectively, the camera and flashlight of a mobile phone. However, in alternative embodiments of the invention, illustrated herein, the light source may be independent of the phone flash light.

In FIG. 2 the components of FIG. 1 are shown attached to a clip 9 which is sized and shaped to wrap around at least a part of a mobile phone. At least one part of the clip has the components of the imaging system attached thereto and at least another part is attached to the mobile phone. The styling of the clip is such that it, ideally can be used with any shape of mobile phone. In alternative embodiments an adapter is provided that attaches to a mobile phone using other conventional attachments such as adhesive, magnetic links screws and the like.

In FIG. 3 the powering of the imaging system is shown and it can be seen that in a first embodiment light source 4 is powered by an electric cable 10 that is connected to a USB socket 11 using a USB connector 12. Alternatively light source 4 is powered by an electric cable 10 that is connected to an Audio jack 13. In alterative embodiments the powering of the imaging system is undertaken using a battery, ideally a rechargeable battery.

In FIG. 4 it is shown that the control of the imaging system is achieved by a simple switch 14 or control electronics 15 of a conventional nature when used in a conventional fashion.

In FIG. 5 the imaging system is shown with particular reference to how illumination of the retina is optimised. The system is as described with reference to FIG. 1 but in the expanded view at the bottom of the figure it can be seen that prism 5 is provided with at least one, and in this embodiment a number of optical adjustment members in the form of filtering and/or optical absorbing members 16. These members 16 are selectively positioned on/in said prism to manage the direction and intensity that light exits said prism and travels towards eye 7. Additionally, it can also be seen that having regard to the camera/pupil axis, light exiting said prism does so at an angle of approximately 5° and so is effectively off-center. Advantageously, we have discovered that this arrangement ensures optimum, i.e. even, illumination of the retina within the field-of-view of the imaging system, please see FIGS. 13 and 14.

In FIG. 6 the imaging system is shown with particular reference to how reflection of light from the cornea is blocked or minimised. In this embodiment a circular polariser 17 is positioned in front of prism 5 in the path of light that is directed towards eye 7. Alternatively, as shown in FIG. 7 a pair of circular polarisers with the same polarity are positioned so that one is in the path of light directed towards eye 7 and one is in the path of light reflected from eye 7. Both of these arrangements minimise light reflection from the retina thus improving the imaging characteristics, please see FIG. 12.

In FIG. 8 the imaging system is shown with particular reference to how the retina is illuminated. In the embodiment shown in the upper part of the figure a plurality of light sources are provided, in this illustration two light sources 4, each one with its own light channeling member 3 and prism 5. In another version of the invention the plurality of light sources 4, in this illustration two, may feed into a single channeling member 3 for example of a circular nature such that each light source feeds into a single circular prism. Alternatively, as shown in the lower part of this figure a plurality of light beams may be generated from a single light source 4 by the use of a prism with a multitude of facets thus splitting light from a single source into a multitude of beams.

Alternatively again, as shown in FIG. 9 light source 4 may be a single ring illuminator 18.

In FIGS. 10 and 11 there is shown another embodiment of the invention depicted in FIGS. 1 and 8 wherein light source 4 is substituted for either a light source 22 capable of emitting single or dual wavelengths (e.g. blue, blue-green or RGB LED) or a light source 23 capable of emitting a single wavelength or white LED. Thus the interrogating light is either 19 of a single wavelength (e.g. 465-490 nm) of a diverging light beam or 21 comprises a second single wavelength or is a white diverging light beam.

Similarly, FIGS. 12 and 13 show another embodiment of the invention depicted in FIG. 1 wherein light source 4 is retained but a filter 25 such as a fixed colour filter (e.g. blue-green bandpass) is inserted in the optical path either before light enters prism 3 or when or after it emerges from prism 3. Thus the interrogating light 19 is a single wavelength (e.g. 465-490 nm) diverging light beam.

In FIGS. 10, 12 and 13 emitted light 20 is represented as autofluorescence of the ocular fundus or by a fluorescent dye. In FIG. 11 emitted light 24 is represented as full colour reflection from the ocular fundus in addition to light emitted by autofluorescence of the ocular fundus or by a fluorescent dye.

In FIGS. 14-16 there is shown another embodiment of the invention depicted in FIG. 1 wherein light source 4 is substituted for either a light source 22 capable of emitting single or dual wavelengths (e.g. blue, blue-green or RGB LED) or where wherein light source 4 is retained but a filter 25 such as a fixed colour filter (e.g. blue-green bandpass) is inserted in the optical path either before light enters prism 3 or when or after it emerges from prism 3. In all these embodiments emitted light 26 is represented as the reflection of a selected wavelength or wavelengths from ocular fundus. Thus, in these embodiments the reflected light is of a selected wavelength, or wavelengths, from the ocular fundus.

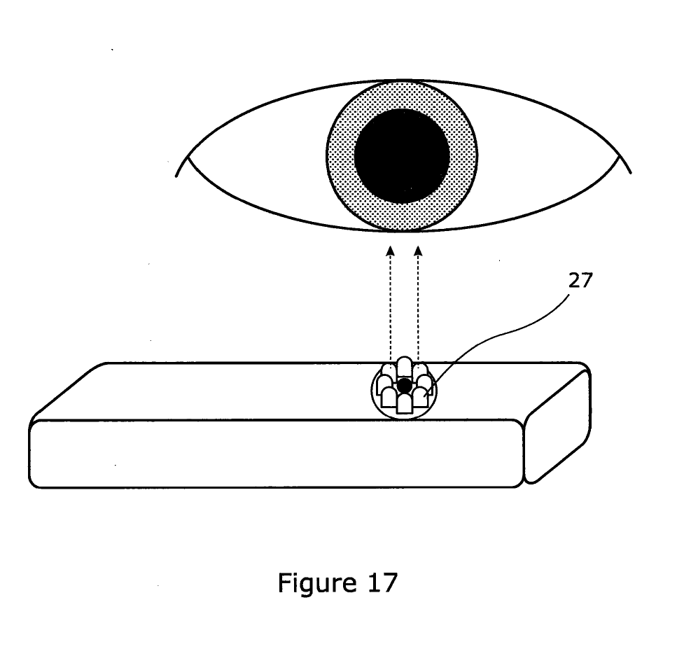

In FIG. 17 there is shown another embodiment of the invention wherein light source 27 is arranged in a circular or eliptical configuration around camera lens 1.

We have tested the above optical arrangements and the results are shown in FIGS. 18-23.

The introduction of a pair of circular polarizers with one placed directly on the output surface of the prism and the other in-front of the camera lens causes a marked reduction in size and intensity of corneal reflection. FIGS. 18a and 18b show the corneal reflection present when no polarizing filters are used. The intensity and size of the reflection is such as to allow significant features on the fundus to be entirely obscured, for example as can be seen in FIG. 18b, the entire optic disc.

By comparison the residual corneal reflection which is left after the introduction of the polarizing filters described is of such size and intensity that it presents no significant obscuration of the fundus image. In fact, even when the residual reflection is directly over a particular structure, as can be seen with the vessel in FIG. 19, the structure can still be seen with satisfactory clarity.

Indeed, if we provide a prism that contains regions that act as polariser and diffuser, rather than applying said polariser and diffuser externally, the image further improves, as shown in FIG. 19b, where the residual halo in FIG. 19 is completely suppressed, while maintaining a full and even illumination of the fundus.

In addition the placement of a diffusing film directly onto the illumination source also allows for a more even illumination of the fundus without effectively increasing the working distance of the device.

As both the illumination source and the camera are laterally separated, a shadow is cast if their optical axes are parallel. The size of this shadow varies as a function of lateral separation of the source and camera, working distance from the eye, difference in the illuminator's and the imaging system's working distance from the eye, focusing power of the eye and angle of the eye relative to the source and camera. However, we have found that the shadow can be minimised for the typical/desired values of these variables by tilting the optical axis of the illumination source relative to the camera.

Thus the angle of the illumination sources' optical axis is neither trivial nor a fixed value for every user-case. Whether a positive or negative tilt is required depends on the lateral positioning of the illumination source relative to camera. As such it is only beneficial to give a range of angles from which technical advantage can be derived depending on the illumination source positioning, desired working distance and desired retinal area to be imaged. Despite this fact, advantage can be derived from a tilt within the range of 0-20 degrees, with a tilt between 3-10 degrees being most typical.

An example of how a single source device can use such tilting of the illumination source's optic axis so to minimise the shadow for viewing a particular structure, the optic nerve in this case, is shown in FIG. 20. The subject in this case has not had their eye dilated using dilating drops (i.e. it is a non-mydriatic image). Acquiring such an image without this off axis tilt would be markedly difficult, if not impossible.

How the shadow is reduced through tilting the optical axis of the source can be simulated by placing a hemi-spherical reflector behind an aperture and arranging an illumination source and detector with a small lateral separation. Results of a simulation are shown in FIGS. 21 and 22. It should be noted that the shadowing for these particular angles is only representative of a specific lateral separation and working distance.

Similarly increasing the separation of the source and the imaging system in the direction of the imaging system's optical axis reduces shadowing by increasing the illuminator's field-of-view relative to the imaging system's field-of-view. This is of particular advantage where it is possible to place the illuminator closer to the eye than it is to place the imaging system, for example a smartphone camera whose working distance is limited by the size and geometry of the phone in which it is integrated, thus reducing shadowing by increasing the field-of-view of the illuminator without decreasing the field-of-view of the imaging system.

Indeed, in FIG. 23 there is shown:
(a) a configuration with the channelling member's centre being coplanar with detector optics' front face;
(b) a false-greyscale image of the illumination distribution on the fundus of a normal adult eye when the configuration in (a) is used.
(c) a false greyscale of the fundus image formed by the detector optics when the configuration in (a) is used.

In an alternative configuration i.e.
(d) a configuration with the channelling member centre being 4mm closer to the eye than the front face of the detector optics.
(e) a false-greyscale image of the illumination distribution on the fundus of a normal adult eye when the configuration in (d) is used. Area is to the same scale as (b)
(f) a false greyscale of the fundus image formed by the detector optics when the configuration in (d) is used. Area is to the same scale as (c).

The viewed retina or images or videos of the retina can be used, for example, to calculate the optic nerve cup to disc ratio (an important diagnostic parameter), optic nerve head size, determine optic nerve abnormalities, retinal vessel calibre and tortuosity as measures of systemic diseases such as hypertension, detection of retinal anomalies inclusive of but not limited to the presence or absence of drusen/choroidal neovascular membrane, and exudates/cotton wool spots/microvascular abnormalities/new vessel disease, which can aid in the diagnosis of diseases such as macular degeneration and diabetic retinopathy, respectively. Other ophthalmic and systemic conditions visible in the retina using the device include, but are not limited to: malaria retinopathy, retinopathy of prematurity, retinitis pigmentosa, retinoblastoma, choroidal melanomas, other eye tumours, macular dystrophies/degenerations, retinal detachment/retinoschisis, optic neuropathies, macular hole, retinal vessel occlusions (artery/vein/tributaries), genetic conditions of the eye.

Finally, it can be advantageous to adjust the intensity of light stimulating the eye or the field diameter of the light stimulating the eye in order to allow a consistent pupil size to be maintained during non-mydriatic use where the pupil diameter is not fixed pharmacologically. Such an adjustment is advantageous to image particular structures of the eye or for a particular field-of-view (e.g. the optic nerve typical requires approximately 5 degree field-of-view). This field of view varies with pupil diameter which, at a given photopic luminance and field diameter, vary from person to person, primarily as a function of age. To this end, the adapter is provided with a control device allowing a user to vary the stimulating photoptic luminance between 10 and 10,000 candela per metre squared, but most typically between 200 and 4000 candela per metre squared, or with the ability to adjust the luminance delivered across a scale of discrete selected units.

Those skilled in the art will appreciate that the said control device may reduce the delivered luminance by extinguishing or dimming one or more light sources, moving one or more light sources mechanically, introducing or moving optical components so as to change the direction, intensity or dispersion of the light directed towards the eye, or to direct one or more sources away from the eye altogether. Conversely, those skilled in the art will appreciate that the delivered luminance may be increased by lighting or increasing the brightness of one or more light sources, moving one or more light sources mechanically or by introducing optical components so as to change the direction, intensity or dispersion of the light directed towards the eye, or to guide one or more sources towards the eye where said source had previously been directed away from the eye.

In this context FIG. 24 shows an image acquired using two sources of the same intensity on a non-mydriatic eye. The field-of-view acquired is only just that at which the optic nerve can be viewed, however, FIG. 25 shows an image acquired using the same device that was used to take the image shown in FIG. 24, but with one of the two sources disabled. The pupil of the eye is larger than in FIG. 24 and therefore more of the fundus is visible around the optic nerve.

In the alternative, or even additionally, the adapter is configured to include a light source, or sources, that are pulsed or, otherwise vary in intensity with time, in order to delay the pupillary reflex.

1. An adapter for modifying a mobile communication device to create an ophthalmoscope or fundus camera for ophthalmoscopy or fundus imaging, respectively, comprising:
at least one illumination device for illuminating an eye of an individual prior to viewing same or taking at least one image of same; wherein said illumination device comprises
at least one light channeling member adapted for directing light into the eye to be imaged, wherein said light channeling member comprises
at least one prism having contained therein or provided thereon at least one optical adjustment member.

2. The adapter according to claim 1 wherein said optical adjustment member comprises one or more regions adapted to perform at least one of the following functions: to act as a diffuser; to act as an absorber; or to act as a polarizer.

3. The adapter according to claim 1, wherein said light channeling member comprises a plurality of prisms.

4. The adapter according to claim 1, wherein said illumination device comprises a light source.

5. The adapter according to claim 4, wherein said light source is positioned off-center with respect to a pupil of the eye to be imaged and/or an axis of retinal imaging optics.

6. The adapter according to claim 5 wherein said light source is off-center by an amount selected from the group comprising: 0.1 mm-4 mm, including all 0.1 mm intervals there between.

7. The adapter according to claim 1, wherein said light source is adapted to emit a divergent, non-focused illumination beam.

8. The adapter according to claim 1, wherein said light source is on an axis tilted away from the optical imaging axis by an amount selected from the group comprising: 1-20 degrees, including all 0.01 intervals there between.

9. The adapter according to claim 1, wherein said prism further comprises at least one reflective member positioned so that light exiting from said prism is reflected towards said eye.

10. The adapter according to claim 1, wherein said light source comprises a waveguide with at least one opening positioned so that, in use, light exiting from said waveguide is directed via said prism towards said eye.

11. The adapter according to claim 10 wherein said waveguide is provided with at least one reflective member whereby light exiting from said waveguide is reflected via said prism towards said eye.

12. The adapter according to claim 10, wherein said waveguide is provided with a scattering structure or a re-emissive structure.

13. The adapter according to claim 1, wherein said illumination device comprises, either as part of said optical adjustment member and/or independently thereof, at least one circular polarizer positioned in the path of light that has to enter the eye.

14. The adapter according to claim 13 wherein a second circular polariser is provided and positioned to block any specular reflection.

15. The adapter according to claim 14 wherein said second circular polariser has the same or substantially the same polarisation chirality as the illumination polarizer.

16. The adapter according to claim 1, wherein said illumination device is either a flash of the mobile communication device, or an independently powered light source.

17. The adapter according to claim 1, wherein a plurality of light sources is provided and arranged so at to illuminate the eye from a plurality of different directions.

18. The adapter according to claim 17 wherein a plurality of light channeling members are provided or a single multi-faceted light channeling member is provided that conveys light from different directions simultaneously.

19. The adapter according to claim 1, wherein the illumination device comprises an illuminator ring or a circular or linear or curved illumination segment provided around the camera.

20. The adapter according to claim 1, wherein the illumination device or light source is separated from the imaging system in the direction of the imaging system's optical axis.

21. The adapter according to claim 1, wherein the adapter is provided with a control device so that the intensity of light stimulating the eye or the field diameter of the light stimulating the eye can be adjusted.

22. The adapter according to claim 21 wherein the control device is adapted to allow a user to vary the stimulating photoptic luminance between 10 and 10,000 candela per metre squared, including all one unit intervals there between.

23. The adapter according to claim 21 wherein the control device is adapted to allow a user to vary the stimulating photoptic luminance between 200 and 4000 candela per metre squared, including all one unit intervals there between

24. The adapter according to claim 21 wherein the control device is adapted to allow a user to adjust the luminance delivered across a scale of discrete units.

25. The adapter according to claim 4, wherein said light source is adapted to be pulsed or varied in intensity with time.

26. An ophthalmoscope or fundus camera, comprising:
a mobile communication device having an integral camera and at least one associated illumination device for illuminating an eye of a patient prior to viewing same or taking at least one image of same; wherein said illumination device comprises
at least one light channeling member adapted for directing light into the eye to be imaged characterized in that said light channeling member comprises
at least one prism having contained therein or provided thereon at least one optical adjustment member.

27. A method for visualising the retina through the pupil of an individual comprising using the ophthalmoscope according to claim 26.

28. A method for imaging an interior of an eye of an individual comprising, using the fundus camera according to claim 26.

29. (canceled)

30. The method of claim 27, wherein the method: calculates optic nerve cup to disc ratio, determines optic nerve head size, determines optic nerve abnormalities, determines retinal vessel calibre and tortuosity; investigates or diagnoses hypertension, detects retinal anomalies selected from drusen/choroidal neovascular membrane, and exudates/cotton wool spots/microvascular abnormalities/new vessel disease, macular degeneration, diabetic retinopathy, malaria retinopathy, retinopathy of prematurity, retinitis pigmentosa, retinoblastoma, choroidal melanomas, other eye tumours, macular dystrophies/degenerations, retinal detachment/retinoschisis, optic neuropathies, macular hole, retinal vessel occlusions (artery/vein/tributaries) and genetic conditions of the eye.

31. (canceled)
